(19) United States
(12) Patent Application Publication
(10) Pub. No.: US 2019/0069837 A1
(43) Pub. Date: Mar. 7, 2019

(54) BRUXNOSTIC DEVICE

(71) Applicant: THE BOARD OF REGENTS OF THE UNIVERSITY OF TEXAS SYSTEM, Austin, TX (US)

(72) Inventor: Shawn S. Adibi, Houston, TX (US)

(21) Appl. No.: 16/121,329

(22) Filed: Sep. 4, 2018

Related U.S. Application Data
(60) Provisional application No. 62/553,444, filed on Sep. 1, 2017.

(51) Int. Cl. A61B 5/00

(52) U.S. Cl. CPC A61B 5/4557; A61B 2562/0233; A61B 2562/0247

(57) ABSTRACT

A bruxnostic device includes a plastic channel configured to be received over a plurality of teeth of an individual. The bruxnostic device further comprises a layer of pressure-indicating material enclosed within the plastic channel in a position to be disposed between maxillary teeth and mandibular teeth of the individual when the plastic channel is received over the plurality of teeth. The pressure-indicating layer produces a color indication in response to pressure between the maxillary teeth and the mandibular teeth.

### STATEMENT OF RELATED APPLICATIONS

This application depends from and claims priority to U.S. provisional application Ser. No. 62/553,444 filed on Sep. 1, 2017, which is incorporated by reference as if fully set forth herein.

### FIELD OF THE INVENTION

This application relates to methods and apparatus for diagnosing or evaluating bruxism.

### BACKGROUND

Bruxism is a condition that causes unconscious teeth grinding. Some patients may grind their teeth independent of whether they are awake or asleep. However, teeth grinding during sleep is of great concern, since the patient cannot actively stop themselves from grinding their teeth. Untreated, bruxism can lead to destruction of expensive dental procedures due to the high forces applied by the jaw. Teeth grinding can also cause a loss of enamel from the teeth, which may then lead to further detrimental effects including tooth decay, dental implant cracks, and tooth or jaw pain. Patients are typically unaware of their condition and may experience significant tooth wear prior to an official diagnosis. However, there are currently no effective diagnostic tools that allow dental healthcare providers to determine the presence and severity of bruxism in their patients. Therefore, dental practitioners often administer questionnaires as a way to screen patients for potential bruxism and may use X-rays to determine the degree of tooth wear and/or extent of the damage.

Physiological, day-to-day bite force ranges from 30-130 kg/cm². Based on interviews conducted with dental practitioners and orthodontists, pathological bite force for a bruxism diagnosis can be considered any force exceeding 150 kg/cm². A simple intraoral device, such as a mouthguard, mouth splint or other occlusal guard, may be effective to spread out this pressure across the jaw and create a physical barrier between the upper and lower teeth. For example, a typical mouthguard is made of plastic or rubber and can be customized to fit the mouth of a patient. While these devices may help to prevent further damage to the teeth, a late diagnosis of bruxism may require a patient to replace molar caps, replace implants, contribute to temporomandibular disorders, or undergo expensive procedures.

### SUMMARY OF THE PRESENT INVENTION

One embodiment provides a bruxnostic device comprising a plastic channel configured to be received over a plurality of teeth of an individual, wherein the plurality of teeth are selected from maxillary teeth, mandibular teeth, and a combination thereof. The bruxnostic device further comprises a layer of pressure-indicating material enclosed within the plastic channel in a position to be disposed between the maxillary teeth and the mandibular teeth of the individual when the plastic channel is received over the plurality of teeth, wherein the pressure-indicating layer produces a color indication in response to pressure between the maxillary teeth and the mandibular teeth.

### BRIEF DESCRIPTION OF THE DRAWINGS

FIG. 1 is a plan view of an embodiment of a bruxnostic device.

FIG. 2 is a side view of the bruxnostic device of FIG. 1.

FIG. 3 is a cross-sectional view of the bruxnostic device of FIGS. 1 and 2 taken along line 3-3 shown in FIG. 2.

FIG. 4 is a schematic plan view of the bruxnostic device of FIGS. 1-3 having a pressure-indicating layer that extends over a single undivided section.

FIG. 5 is a schematic plan view of the bruxnostic device of FIGS. 1-3 having a pressure-indicating layer that is divided into multiple sections.

FIGS. 6A and 6B provide a side view and a cross-sectional view of an embodiment of a bruxnostic workpiece including a first vacuum-formed layer of thermal forming material.

FIGS. 7A and 7B provide a side view and a cross-sectional view of the bruxnostic workpiece of FIGS. 6A and 6B including an orthodontic resin applied to the first vacuum-formed layer to form a surface.

FIGS. 8A and 8B provide a side view and a cross-sectional view of the bruxnostic workpiece of FIGS. 7A and 7B including a pressure-indicating layer applied to the surface.

FIGS. 9A and 9B provide a side view and a cross-sectional view of a finished bruxnostic device of FIGS. 8A and 8B including the bruxnostic workpiece with a second vacuum-formed layer of thermal forming material applied to isolate the pressure-indicating layer from any fluids that may come into contact with the bruxnostic device.

### DETAILED DESCRIPTION OF PREFERRED EMBODIMENTS

One embodiment provides a bruxnostic device comprising a plastic channel configured to be received over a plurality of teeth of an individual, wherein the plurality of teeth are selected from maxillary teeth, mandibular teeth, and a combination thereof. The bruxnostic device further comprises a layer of pressure-indicating material enclosed within the plastic channel in a position to be disposed between the maxillary teeth and the mandibular teeth of the individual when the plastic channel is received over the plurality of teeth, wherein the pressure-indicating layer produces a color indication in response to pressure between the maxillary teeth and the mandibular teeth.

In one embodiment of the bruxnostic device of the present invention, the plastic channel may be, without limitation, a thermoplastic material or an elastomeric material. In one embodiment of the bruxnostic device of the present invention, the plastic channel includes a major portion of ethylene-vinyl acetate. Furthermore, the plastic channel is preferably form fit to contours of the plurality of teeth and is preferably clear to allow visual observation of the color indication produced in the enclosed pressure-indicating layer. Optionally, an embodiment of the bruxnostic device may include a deeper channel formed by extending the inward-facing wall and the outward-facing wall so that the extended portion of the inward-facing wall and the extended portion of the outward-facing wall engage the gingiva.

The pressure-indicating material enclosed within an embodiment of the bruxnostic device of the present invention may produce the color indication in response to an applied pressure exceeding a predetermined pressure threshold. The color indication may be a color intensity that varies in proportion to an amount of the pressure applied to the pressure-indicating material. For example, but not by way of limitation, the color intensity may vary in proportion to the amount of the applied pressure between a predetermined minimum pressure and a predetermined maximum pressure.

In various embodiments of the bruxnostic device of the present invention, the layer of pressure-indicating material is a pressure-indicating film. The pressure-indicating film is preferably disposed generally perpendicular to a central axis of the plastic channel. Embodiments of the bruxnostic device may include various pressure-indicating films that produce a color indication over different pressure ranges. For example, a first embodiment of the bruxnostic device of the present invention may have a pressure-indicating film that produces a color indication with varying color intensity at applied pressures between 0 and 150 kg/cm², a second embodiment of the bruxnostic device of the present invention may have a pressure-indicating film that produces a color indication with varying color intensity at applied pressures between 150 and 300 kg/cm², and a third embodiment of the bruxnostic device of the present invention may have a pressure-indicating film that produces a color indication with varying color intensity at applied pressures pressure greater than 300 kg/cm². Each embodiment of the bruxnostic device of the present invention may be identified with a chart exhibiting a predetermined association between color intensity and the applied pressure required to produce same. Accordingly, the color intensity exhibited by an embodiment of the bruxnostic device may be visually observed and then used as an index into the chart to identify and to correlate the applied pressure or applied pressure range associated with the observed color intensity. Embodiments of the bruxnostic device allow dental practitioners to identify the presence and severity of bruxism in patients before they must undergo expensive ortho-prosthesis or related dental procedures.

In one non-limiting example, an embodiment of the bruxnostic device of the present invention may be prepared using an additive process, wherein various layers are sequentially applied to an initial base layer. For example, but not by way of limitation, the base layer may be a thermal forming material that has been vacuum-formed over a mold of a patient's teeth. A second layer may be an orthodontic resin applied into crevices of the base layer in order to form a more crevice-free surface. Next, a third layer may be a pressure-indicating material, such as a pressure-indicating film. Then a fourth layer may be a thermal forming material that extends over the second and third layers and is secured to the base layer. The fourth layer may be used to seal the enclosed third layer and/or the second and third layers between the first layer and the fourth layer to enclose the third layer and/or the second and third layer within the channel.

In another specific example of an embodiment of the bruxnostic device of the present invention, the base layer may be made of Buffalo Brand Thermal Forming Material or an equivalent material. Alternately, an embodiment of the bruxnostic device of the present invention may include a base layer of a Sof-Tray® classic sheets forming material made by Ultradent or an equivalent material. The stiffness of these materials may vary. In some embodiments of the bruxnostic device of the present invention, the stiffer materials provide for better retention of the bruxnostic device onto the top teeth. The second layer may be a Hygenic Brand Orthodontic Resin that fills in the ridges and/or crevices of the base layer that reflect the contour of the patient's teeth. In this manner, the orthodontic resin creates an even, or more ridge-free or crevice-free, surface for adhering a third layer. The third layer may include a pressure-sensing film disposed along the even, or more ridge-free or crevice-free, surface of the second layer. Furthermore, the third layer may also include Bondic Brand Plastic Welder Glue applied to the sides of the base layer to ensure uniform and reliable adherence of a fourth layer. Bondic Brand Plastic Welder Glue may be used to secure the sensor on the base layer. The fourth and final layer may be a Sof-Tray® Classic Sheets Forming Material from Ultradent. Using the Sof-Tray® Classic Sheets Forming Material from Ultradent for the final layer provides comfort for the patient's tongue when wearing the bruxnostic device.

In a further specific embodiment, a bruxnostic device of the present invention may be constructed using a pressure-indicating film layer and mouthguard layers made of thermoplastic poly (vinyl acetate-ethylene) copolymer thermal forming material. A vacuum former apparatus may be used to create a mouthguard mold of a patient's upper teeth using 0.5 mm thick Buffalo Brand Thermal Forming Material, or an equivalent material, or using 0.25 mm-0.9 mm of Sof-Tray® Classic Sheet(s) forming material from Ultradent Products, Inc. of South Jordan, Utah, or other substantially similar material. Hygenic Brand Orthodontic Resin may be carefully applied to level out the grooves created by the teeth and the resin may then be left to cure. A thin layer of Fujifilm Prescale®-Nip Pressure Indicating Sensor Film may then be placed over the leveled-out surface of orthodontic resin, since that is the area most subject to bruxism damage. The layer of pressure-indicating film may be secured to the orthodontic resin using a thin layer of Bondic Brand Plastic Welder Glue that was cured with ultraviolet light. Lastly, a layer of 0.25 mm-0.9 mm Sof-Tray® classic sheets forming material from Ultradent can be vacuum-formed over the pressure-indicating film, orthodontic resin and part of the outward-facing surface of the base layer to create a protective layer, preventing the teeth or fluids from coming into contact with the film or adhesive.

In yet another specific embodiment of the bruxnostic device of the present invention, the base layer may be made of Sof-Tray® Brand from Ultradent (0.25 mm-0.9 mm) to which is applied a thin layer of Bondic Brand Plastic Welder Glue that was cured with ultraviolet light and a thin layer of Fujifilm Prescale®-Nip Pressure Indicating Sensor Film may then be placed over the Bondic layer and covered with a layer of about 0.25 mm-0.9 mm Sof-Tray® classic sheets forming material that can be vacuum formed over the pressure-indicating film, orthodontic resin and part of the outwardly facing surface of the base layer to create a protective layer, preventing the teeth or fluids from coming into contact with the film or adhesive. These layers are then sealed.

The Fujifilm Prescale®-Nip Pressure Indicating Sensor Film includes a developer sheet and a transfer sheet. The transfer sheets contain colored microcapsules. When pressure is applied by the teeth closing on an embodiment of the bruxnostic device of the present invention constructed using such transfer sheets, the heat from the pressure melts the developer sheet and the microcapsules on the transfer sheet become semi-permeable, allowing the developer to enter the microcapsules and produce a color change. The intensity of the color that is produced depends on the amount of pressure applied to the film. As indicated above, a pressure-indicating film may be prepared having a specific applied pressure range for producing the color indication. It is possible to make different pressure-indicating films that each have their own unique activation pressure range. For example, the manufacturer of the foregoing Fujifilm pressure-indicating film makes films having various pressure ranges, such as a “Low range” film that includes separate developer and transfer sheets that provide a color indication at a pressure between 25 and 100 kg/cm², and a “Medium range” film that includes a single sheet having the developer and transfer layers that provide a color indication at a pressure between 100 and 500 kg/cm².

In order to test an individual for bruxism, the individual wears an embodiment of the bruxnostic device of the present invention in the same manner as a mouth guard. The embodiment of the bruxnostic device may be worn over a designated period of time such as, for example, but not by way of limitation, during sleep for from two to seven nights. The exact amount of time and conditions in which the embodiment of the bruxnostic device is worn may vary according to diagnostic objectives and instructions provided by a dental professional. However, when the embodiment of the bruxnostic device is worn, pressure between the upper and lower teeth will cause a color change in only those areas where the pressure has met or exceeded the stated pressure range of the pressure-indicating film. Areas of pressure sensitive film where the inter-occlusal pressure points exceeded the threshold pressure of the film at any time while the bruxnostic device was worn will experience a color change. Accordingly, the embodiment of the bruxnostic device may be considered as making a record of the areas experiencing the threshold pressure during a diagnostic period in which the bruxnostic device is worn.

In one example of a method of determining the severity of bruxism in an individual, the individual may first wear a bruxnostic device including a “Low range” pressure-indicating film. After use, if the Low range bruxnostic device exhibits a color change over an amount of area exceeding some threshold, then the individual may be asked to undergo a secondary test by wearing a Medium range bruxnostic device. The pressure range of the Low range bruxnostic device may be selected to identify mild to moderate bruxism, whereas the pressure range of the Medium range bruxnostic device may be selected to identify moderate to severe bruxism forces. Of note, it is possible that a Low range force reading for small female with small face and thus small masticatory muscles may be a significant finding to aid practitioner in the diagnosis of moderate bruxism, even though a mid-range of force detected in a larger individual may be a finding in consistent with mild bruxism.

For example, a first bruxnostic device might be used to indicate mild bruxism using a pressure-indicating material having a color indication at pressures between 0 and 150 kg/cm². A determination of mild bruxism might be addressed by more rigorous and continued observation. A second bruxnostic device might be used to indicate moderate bruxism using a pressure-indicating material having a color indication at pressures between 150 and 300 kg/cm². A determination of moderate bruxism might be addressed by use of night guard (i.e., a mouth guard worn at night). Still further, a third bruxnostic device might be used to indicate severe bruxism using a pressure-indicating material having a color indication at pressure greater than 300 kg/cm². A determination of severe bruxism might be addressed by treatment with medications and or (psycho)therapeutic intervention.

In one mode of treatment using an embodiment of the bruxnostic device of the present invention, the individual may wear a first bruxnostic device for several nights before returning the device to a dental practitioner. The practitioner may review the color indication exhibited by the first bruxnostic device, record the color indication of the bruxnostic device with a photo or digital image, and observe several areas of the individual's teeth where the bruxnostic device experienced a maximal force as indicated by a maximum color indication. The practitioner might then decide to investigate further by providing the individual with a second bruxnostic device with instructions to wear the second bruxnostic device for several nights before returning the device. The practitioner may then review and record the color indication exhibited by the second bruxnostic device and observe several areas of the individual's teeth where the second bruxnostic device experienced the greatest force as indicated by the greatest color indication.

Although embodiments of the bruxnostic device of the present invention may use a single continuous layer of pressure-indicating material or continuous sheet of pressure sensitive film, embodiments may also use multiple separate regions or multiple separate sheets of film positioned across various regions or zones of interest. Still, the multiple regions or sheets may be positioned edge-to-edge or spaced apart, either to facilitate manufacturing or to improve detection of bruxism forces.

Embodiments of the bruxnostic device of the present invention are preferably designed as maxillary bruxnostic devices to be worn on the upper teeth, but may alternately be designed as mandibular bruxnostic devices to be worn on the lower teeth. Individual tolerance and compliance may be better with the maxillary bruxnostic device. It is also possible that an individual might wear a mandibular bruxnostic device and a maxillary bruxnostic device, either simultaneously or sequentially. Various approaches may be used to more precisely identify points of contact between the upper and lower teeth, and the amount of pressure associated with those points of contact.

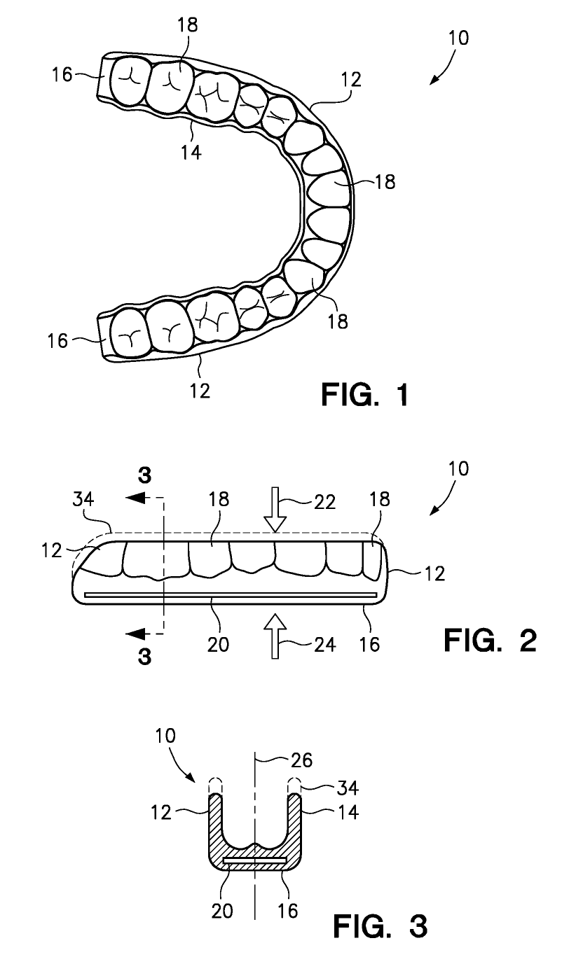

FIG. 1 is a plan view of an embodiment of a bruxnostic device 10 of the present invention. The bruxnostic device 10 is generally U-shaped channel that is received about an individual's teeth. As shown, the U-shaped channel has an outward-facing wall 12 that extends upward along the front of the teeth, an inward-facing wall 14 that extends upward along the back of the teeth, and a base 16 that extends between the outward-facing and inward-facing walls. The walls and base are preferably contoured to the teeth, for example by vacuum forming or pressure laminating, such that customized impressions 18 are formed for each of the teeth. In reference to FIG. 1, the term “upward” is used in reference to the base 16 as shown. However, the bruxnostic device 10 may be used with maxillary (upper) teeth or mandibular (lower) teeth without departing from the same general description of the device. It should be appreciated that the orientation of the bruxnostic device will vary according to the application as a maxillary device (as shown) or mandibular device (inverted from the position shown), and the position of the individual. Furthermore, the walls and base of the bruxnostic device may be custom trimmed for the size of the individual's mouth, the individual's comfort, and the area of interest for determine the presence and severity of bruxism.

FIG. 2 is a side view of the bruxnostic device 10 of FIG. 1. The outward-facing wall 12 is seen, but portions of certain tooth impressions 18 are also shown formed into the wall 12 and base 16. Furthermore, a layer of pressure-indicating material 20 is shown enclosed or embedded within the base 16 below the tooth impressions 18. When worn by an individual, for example on their maxillary (upper) teeth, any teeth grinding (bruxism) will manifest itself as a downward force (see directional arrow 22) of the maxillary teeth and an upward force (see directional arrow 24) of the mandibular teeth. With the layer of pressure-indicating material 20 disposed between the upper and lower teeth, the downward force 22 and upward force 24 will cause pressure on the material 20. As previously described, this pressure causes the material to exhibit a localized color indication at any point where the upper and lower teeth impart a pressure exceeding a predetermined minimum pressure threshold of the material. In certain embodiments, the color indication may be a color intensity that varies in proportion to an amount of the pressure applied to the pressure-indicating material between a predetermined minimum pressure and a predetermined maximum pressure. The dashed line 34 in FIG. 2 indicates that the outward-facing wall 12 and/or the inward-facing wall 14 may be extended to engage the gingival line or may extend beyond the gingival line to engage the gingiva. The extended portions can be customized as to shape and thickness and length to provide flexure for conformance to the gingiva, to provide stiffness to promote retention, or to provide for a seal with the gingiva.

FIG. 3 is a cross-sectional view of the bruxnostic device 10 taken along line 3-3 shown in FIG. 2. The outward-facing wall 12 and the inward-facing wall 14 extend upwardly from the base 16. The layer of pressure-indicating material 20 is enclosed within the base 16 and is disposed generally perpendicular to a central axis 26 of the U-shaped channel. In this view, it is clear that the pressure-indicating material 20 is disposed to be directly between the maxillary and mandibular teeth when the bruxnostic device 10 is being worn by an individual. Note that the pressure-indicating material 20 will be disposed directly between the maxillary and mandibular teeth regardless of whether the bruxnostic device 10 is being worn as a maxillary bruxnostic device or as a mandibular bruxnostic device. However, the impressions are preferably customized to the maxillary or mandibular teeth, such that any individual bruxnostic device would only be used as either a maxillary bruxnostic device or as a mandibular bruxnostic device. As stated in connection with FIG. 2, the dashed line 34 in FIG. 3 illustrates the optional extension of the outward-facing wall 12 and/or the inward-facing wall 14.

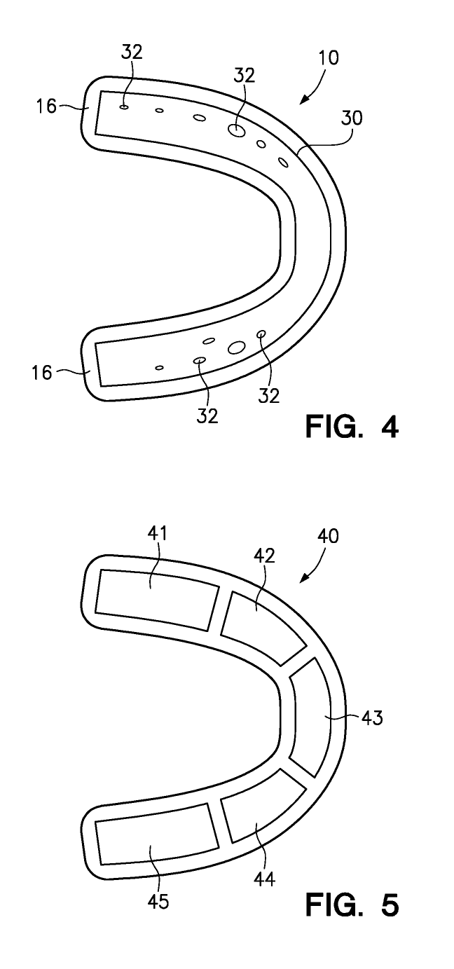

FIG. 4 is a schematic plan view of an embodiment of the bruxnostic device 10 having a pressure-indicating layer 30 that extends over a single undivided section of the base 16. Note that the pressure-indicating layer 30 illustrates a number of color indications 32 as the result of pressure applied to the layer. The color indications 32 indicate the location of an individual's tooth or teeth that have applied a pressure exceeding the minimum pressure threshold of the pressure-indicating material. For example, the larger and darker color indications or spots indicate areas of greater pressure. A dental practitioner may then examine the individual's teeth in those areas to determine an extent of tooth wear or other symptoms of bruxism. The dental practitioner may also determine whether to have the individual wear another bruxism having a pressure-indicating material that exhibits a color indication at a higher pressure or higher range of pressures.

FIG. 5 is a schematic plan view of an alternate embodiment of a bruxnostic device 40 having a pressure-indicating layer that is divided into multiple sections 41-45. The use of separate sections of the pressure-indicating material may facilitate manufacturing, increase durability of the resulting bruxnostic device, or enable the use of different pressure-indicating material in one or more of the sections.

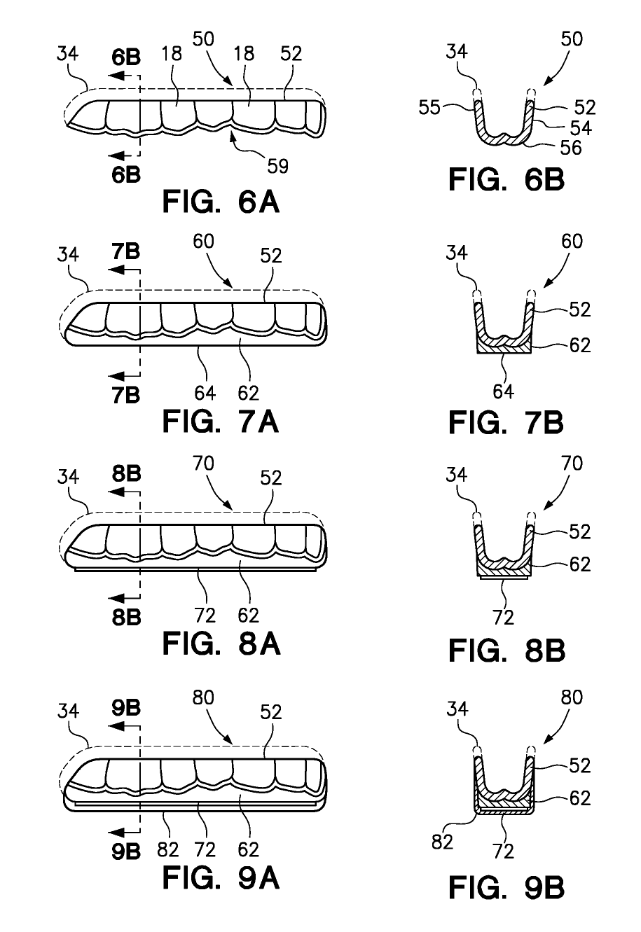

FIGS. 6A and 6B provide a side view and a cross-sectional view, respectively, of a bruxnostic workpiece 50 including a first vacuum-formed layer of a thermal forming material 52. By using a vacuum-forming apparatus, the thermal forming material is heat softened and then pulled firmly against a rigid mold of an individual's teeth. Note the ridges 59 that are formed between adjacent tooth impressions 18 in FIG. 6A. In reference to FIG. 6B, the base 56, outwardly-facing wall 54 and inwardly-facing wall 55 are each shown. The dashed line 34 in each of FIG. 6A and FIG. 6B illustrates the optional extension of the outward-facing wall 12 and/or the inward-facing wall 14.

For example, Buffalo brand thermal forming material or a substantially equivalent material, or Sof-Tray® classic sheets forming material (about 0.5 mm thick, for example) was placed within an AW Pro Dental Vacuum Forming Machine to be heated and a rigid mold of an individual's teeth was placed on a lower platform of the machine. Then, the heat was turned on and when the Buffalo, Sof-Tray® classic sheets forming material or a substantially equivalent material had been melted enough to be concave, the heat was turned off and the vacuum was turned on. The top platform was pulled down and held for ten seconds. Once the material cooled to form a U-shaped channel, the mold was taken out of the channel and the channel was trimmed at the gingival line with scissors and a utility knife.

The “Buffalo Brand Thermal Forming Material” is a clear sheet of ethylene-vinyl acetate (EVA) with dimensions of 0.020 inches thickness, 5 inches width and 5 inches length. This material is available from Buffalo Dental Manufacturing Co., Inc. of Syosset, N.Y. Similarly, information for Sof-Tray® classic sheets forming material is available at the Ultradent website, specifically, at the webpage at https://www.ultradent.com/en-us/Dental-Products-Supplies/Tooth-Whitening/whitening-tray-fabrication/Sof-Tray-Classic-Sheets-Material-for-Vacuum-Forming-of-Trays/Pages/default.aspx. This material is available from Ultradent, 505 West Ultradent Drive (10200 S), South Jordan, Utah 84095-3942.

FIGS. 7A and 7B provide a side view and a cross-sectional view, respectively, of a bruxnostic workpiece 60 including the vacuum-formed thermal forming material 52 of FIGS. 6A and 6B. Hygenic brand orthodontic resin 62 was applied to the first vacuum-formed material 52 to fill in the ridges in the material 52. The resin 62 may be was later sanded down to form a flattened surface 64. The dashed line 34 in each of FIG. 7A and FIG. 7B illustrates the optional extension of the outward-facing wall 12 and/or the inward-facing wall 14.

The Hygenic brand orthodontic resin is an acrylic resin based on a combination of methyl methacrylate and ethylene dimethacrylate. This resin is available from Coltene Whaleden, 235 Ascot Parkway, Cuyahoga Falls, Ohio 44223.

FIGS. 8A and 8B provide a side view and a cross-sectional view, respectively, of an embodiment of a bruxnostic device 70 including the vacuum-formed thermal forming material 52 and the orthodontic resin 62 of FIGS. 7A and 7B. A pressure-indicating film 72 was then bonded to the flattened surface 64 of the orthodontic resin 62 using Bondic Plastic Weld Glue, which was then cured with ultraviolet light. The pressure-indicating film 72 is Fujifilm Prescale® brand Nip Pressure Indicating Sensor Film available from Fujifilm North America Corporation of Valhalla, N.Y. The “Low”, “Medium” and “High” pressure sensitivity films are preferred since the pressure ranges that produce a color indication in those films are relevant to a diagnosis of bruxism while also indicating a severity of the problem. The dashed line 34 in each of FIG. 8A and FIG. 8B illustrates the optional extension of the outward-facing wall 12 and/or the inward-facing wall 14.

FIGS. 9A and 9B provide a side view and a cross-sectional view, respectively, of an embodiment of a finished bruxnostic device 80 including the vacuum-formed thermal forming material 52, the orthodontic resin 62, and the pressure-indicating film 72 of FIGS. 8A and 8B. The bruxnostic device 70 was placed back on the vacuum-forming apparatus, and 0.9 mm thick layer of Sof-Tray thermal forming material 82 was vacuum-formed over the pressure indicating film 72 to isolate the pressure-indicating film 72 from any tissue or fluids that may come into contact with the bruxnostic device 80 while being worn by an individual. The excess Sof-Tray classic sheets material was trimmed, resulting in the finished bruxnostic device 80. The dashed line 34 in each of FIG. 9A and FIG. 9B illustrates the optional extension of the outward-facing wall 12 and/or the inward-facing wall 14.

The Sof-Tray Classic Sheets Brand Thermal Forming Material is a sheet of ethylene-vinyl acetate (EVA) material having dimensions of 0.060 inches thickness, 5 inches width and 5 inches length. In addition to Ultradent, this material, or a substantially equivalent material, may also available from Henry Schein, Inc. of Melville, N.Y.

While some specific materials and the sources from which they are available are disclosed herein above, this is not limiting of the present invention and other comparable materials, and sources thereof, may be substituted without loss of the benefit of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the invention. The word “enclosed,” as that word is used herein, means contained within, embedded within, incorporated within, and/or held within.

The corresponding structures, materials, acts, and equivalents of all means or steps plus function elements in the claims below are intended to include any structure, material, or act for performing the function in combination with other claimed elements as specifically claimed. The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

We claim:

1. A bruxnostic device, comprising:
a plastic channel configured to be received over a plurality of teeth of an individual, wherein the plurality of teeth is selected from the maxillary teeth, the mandibular teeth, and a combination thereof; and
a layer of pressure-indicating material enclosed within the plastic channel in a position relative to the channel to be disposed between the maxillary teeth and the mandibular teeth of the individual when the plastic channel is received over the plurality of teeth;
wherein the layer of pressure-indicating material produces a color indication in response to applied pressure between the maxillary teeth and the mandibular teeth.

2. The bruxnostic device of claim 1, wherein the layer of pressure-indicating material is a pressure-indicating film.

3. The bruxnostic device of claim 2, wherein the pressure-indicating film is disposed generally perpendicular to a central axis of the plastic channel.

4. The bruxnostic device of claim 1, where the pressure-indicating material produces the color indication in response to pressure exceeding a predetermined pressure threshold.

5. The bruxnostic device of claim 1, wherein the color indication is a color intensity in proportion to an amount of the pressure applied to the pressure-indicating material.

6. The bruxnostic device of claim 5, wherein the color intensity varies in proportion to the amount of the applied pressure between a predetermined minimum pressure and a predetermined maximum pressure.

7. The bruxnostic device of claim 1, wherein the plastic channel is a thermoplastic material.

8. The bruxnostic device of claim 1, wherein the plastic channel is an elastomeric material.

9. The bruxnostic device of claim 1, wherein the plastic channel includes a major portion of ethylene-vinyl acetate.

10. The bruxnostic device of claim 1, wherein the plastic channel is form fit to contours of the plurality of teeth.

11. The bruxnostic device of claim 1, wherein the plastic channel is clear to allow visual observation of the color indication produced in the enclosed pressure-indicating layer.

12. The bruxnostic device of claim 2, wherein the pressure-indicating material produces a color indication with varying color intensity at applied pressures between 0 and 150 kg/cm².

13. The bruxnostic device of claim 2, wherein the pressure-indicating material produces a color indication with varying color intensity at applied pressures between 150 and 300 kg/cm².

14. The bruxnostic device of claim 2, wherein the pressure-indicating material produces a color indication with varying color intensity at applied pressures pressure greater than 300 kg/cm².

15. The bruxnostic device of claim 1, wherein the layer of pressure-indicating material is divided into a plurality of separate regions of pressure-indicating material.
